(19) United States
(12) Patent Application Publication
(10) Pub. No.: US 2022/0145252 A1
(43) Pub. Date: May 12, 2022

(54) METHODS FOR MANUFACTURING T CELLS EXPRESSING OF CHIMERIC ANTIGEN RECEPTORS AND OTHER RECEPTORS

(71) Applicant: City of Hope, Duarte, CA (US)

(72) Inventors: Christine E. Brown, Duarte, CA (US); Dongrui Wang, Duarte, CA (US); Stephen J. Forman, Duarte, CA (US)

(21) Appl. No.: 17/599,786

(22) PCT Filed: Mar. 30, 2020
(86) PCT No.: PCT/US2020/025745

§ 371 Date: Sep. 29, 2021

Related U.S. Application Data
(60) Provisional application No. 62/826,884, filed on Mar. 29, 2019.

(51) Int. Cl. C12N 5/0783

(52) U.S. Cl. CPC C12N 5/0636; C12N 2501/2302; C12N 2501/2315; C12N 2510/00; C12N 2501/515; C12N 2501/599; C12N 2501/505

(57) ABSTRACT

A method for manufacturing T cell populations enriched for cells expressing CD27 and useful in T cell therapy is described. The T cell populations are also useful for a variety of purposes requiring a highly active, long-lived T cell population. Such cells elicit a superior antitumor immune response in vitro and in vivo.

### BACKGROUND

Adoptive T cell therapy (ACT) utilizing ex vivo expanded autologous and allogeneic T cells is an attractive therapeutic approach for the treatment of viral infection, cancer and autoimmune disease. Methods that enable the rapid generation of large numbers of therapeutic T cells are critical to the potency and safety of ACT. Various T cell enrichment methods, including selection of defined T cell subsets, as well as expansion methods have been used for ACT. It is desirable to employ a T cell population that permits relatively high activity in vivo and relatively high proliferation potential.

### SUMMARY

Described herein is a method for manufacturing T cell populations useful in T cell therapy, for example, T cells expressing a recombinant T cell receptor (e.g., a chimeric antigen receptor (“CAR”) or T cell receptor (“TCR”)) or tumor infiltrating lymphocytes (“TIL”). The T cell populations are also useful for a variety of purposes requiring a highly active, long-lived T cell population. The methods described herein entail the use of a T cell population enriched for cells expressing CD27. Such cells elicit a superior antitumor immune response in vitro and in vivo. Moreover, effector function after tumor stimulation is better maintained in CD27-enriched CAR T cells.

The enrichment for CD27-expressing T cells can occur before introduction of a transgene expressing a recombinant T cell receptor or after introduction of a transgene expressing a recombinant T cell receptor. Preferably, the T cell population is enriched such that at least 30%, 40%. 50%, 60%, 70%, 80%, 85%, 90%, 95% or more CD27+ T cells. Described herein is a method for preparing a population T cells comprising CD27+ cells expressing a recombinant T cell receptor, comprising: (a) providing a population of human T cells (e.g., PBMC); (b) treating the human T cell population to enrich for CD27+ cells thereby providing a population of T cells that is enriched for CD27+ cells relative to the provided human T cell population; and (c) introducing a nucleic acid molecule expressing a recombinant T cell receptor into the population of cells that are enriched for CD27+ cells relative to the provided T cell population thereby providing population of T cells comprising CD27+ cells expressing a recombinant T cell receptor.

Also described is a method for preparing a population T cells expressing a recombinant T cell receptor, comprising: (a) providing a population of human T cells (e.g., PBMC); (b) introducing a nucleic acid molecule expressing a recombinant T cell receptor into the provided population of human T cells thereby providing a population of T cells expressing a recombinant T cell receptor; and (c) treating the population of T cells expressing a recombinant T cell receptor to enrich the population for CD27+ cells thereby providing a population of T cells comprising CD27+ cells expressing a recombinant T cell receptor.

In various embodiments, the step of providing a population of human T cells comprises: (a) providing a population of human T cells (e.g., PBMC); (b) treating the provided population of human T cells to deplete CD14+ cells and CD25+ cells and thereby providing a population of human T cells; and (c) optionally, treating the population of human T cells to enrich for CD62L+ cells.

In various embodiments of the forgoing methods: the step of providing a population of human T cells does not comprise treating the population of human T cells to deplete CD45RA+ cells; the step of introducing a nucleic acid molecule comprises prior activation of the cells by exposure to an anti-CD3 antibody and an anti-CD28 antibody; the recombinant T cell receptor is a chimeric antigen receptor; the population of T cells comprising CD27+ cells expressing a recombinant T cell receptor comprises at least 30%, 40%, 60%, 70% or 80% CD27+ T cells; the provided population of human T cells comprises no more than 25%, 20%, 15%, 10%, or 5% CD25+ T cells; the provided population of T cells comprises no more than 25%, 20%, 15%, 10%, or 5% CD14+ T cells; the provided population of T cells comprises at least 30%, 40%, 50%, 60%, 70% or 80% CD62L+ cells; the population of T cells comprising CD27+ cells expressing a recombinant T cell receptor comprises no more than 25%, 20%, 15%, 10%, or 5% CD25+ T cells; and the population of T cells comprising CD27+ cells expressing a recombinant T cell receptor comprises no more than 25%, 20%, 15%, 10%, or 5% CD14+ T cells.

Also described is a population of T cells prepared by any of the forgoing the methods. In various embodiments: at least 30%, 40%, 60%, 70% or 80% of the cells are CD27+ cells; less than 5% of the cells are CD25+ and/or less than 5% of the cells are CD14+; greater than 30%, 40%, 60%, 70% or 80% of the human T cells are CD62L+ T cells; and greater than 30%, 40%, 60%, 70% or 80% of the human T cells express a recombinant T cell receptor (e.g., a chimeric antigen receptor); at least 20%, 30%, 40% or 50% of the cells are CD8+; at least 20%, 30%, 40% or 50% of the cells are CD4+; at least 20%, 30%, 40% or 50% of the cells are CD8+ and at least at least 20%, 30%, 40% or 50% of the cells are C48+;

Also described is population of T cells comprising CD27+ cells expressing a recombinant T cell receptor, wherein least 30%, 40%, 60%, 70% or 80% of the cells are CD27+ cells.

In various embodiments: the population comprises no more than 25%, 20%, 15%, 10%, or 5% CD14+ T cells; the population comprises no more than 25%, 20%, 15%, 10%, or 5% CD25+ T cells; the recombinant T cell receptor is a chimeric antigen receptor;

Also described herein is a method of treating a patient, the method comprising administering a composition comprising a population of T cells comprising CD27+ cells expressing a recombinant T cell receptor, wherein at least 30%, 40%, 60%, 70% or 80% CD27+ T cells.

In various embodiments: least 30%, 40%, 50%, 60%, 70% or 80% of the T cells comprise a nucleic acid molecule encoding a recombinant T cells receptor (e.g., a chimeric antigen receptor) and least 30%, 40%, 60%, 70% or 80% of the human T cells are CD27+ T cells; no more than 25%, 20%, 15%, 10%, or 5% of the human T cells are CD14+ T cells; no more than 25%, 20%, 15%, 10%, or 5% of the human T cells are CD25+ T cells; at least 30%, 40%, 60%, 70% or 80% of the human T cells are CD62L+ T cells; at least 30%, 40%, 60%, 70% or 80% of the human T cells comprising a recombinant T cell receptor are CD27+ T cells.

In various embodiment, nucleic acid molecule expressing a recombinant T cell receptor is present in a lentiviral vector or a retroviral vector.

Described herein is a method for preparing a population T cells comprising CD27+ cells expressing a recombinant T cell receptor and for use in a human patient, comprising: providing a population of human PBMC cells; treating the human PBMC cell population to deplete CD14+ cells and CD25+ cells and enrich for CD27+ cells thereby providing a population of T cells that is enriched for CD27+ cells and depleted for CD14+ cells and CD25+ cells relative to an unfractionated human PBMC cell population; and introducing a nucleic acid molecule expressing a recombinant T cell receptor into the population of cells that are enriched for CD27+ cells and depleted for CD14+ cells and CD25+ cells relative to an unfractionated human PBMC cell population thereby providing population of T cells comprising CD27+ cells expressing a recombinant T cell receptor.

Also described is a method for preparing a population T cells expressing a recombinant T cell receptor for use in a human patient, comprising: providing a population of human PBMC cells; treating the human PBMC cell population to deplete CD14+ cells and CD25+ cells thereby providing a population of T cells that is depleted for CD14+ cells and CD25+ cells relative to an unfractionated PBMC cell population; and introducing a nucleic acid molecule expressing a recombinant T cell receptor into the population of cells that is depleted for CD14+ cells and CD25+ cells relative to an unfractionated human PBMC cell population thereby providing a population of T cells expressing a recombinant T cell receptor; and treating the population of T cells expressing a recombinant T cell receptor to enrich the population for CD27+ cells thereby providing population of T cells comprising CD27+ cells expressing a recombinant T cell receptor.

In various embodiments: the step of introducing a nucleic acid molecule comprises prior activation of the cells by exposure to an anti-CD3 antibody and an anti-CD28 antibody; the step of treating to deplete CD14+ cells and CD25+ cells comprises exposing the population of human PBMC to anti-CD14 antibodies and anti-CD25 antibodies; the step of treating to enrich the population for CD27+ cell comprises exposing the population of cells to anti-CD27 antibodies; the recombinant T cell receptor is a chimeric antigen receptor; the population of T cells comprising CD27+ cells expressing a recombinant T cell receptor comprises at least 30%, 40%, 60%, 70% or 80% 85%, 90%, 95% or more CD27+ T cells; the population of T cells comprising CD27+ cells expressing a recombinant T cell receptor comprises no more than 25%, 20%, 15%, 10%, or 5% CD25+ T cells; and the population of T cells comprising CD27+ cells expressing a recombinant T cell receptor comprises no more than 25%, 20%, 15%, 10%, or 5% CD14+ T cells.

Also described is a population of T cells prepared by any of the forgoing methods. In various embodiments: at least 30%, 40%, 60%, 70% or 80% of the cells are CD27+ cells.

Also described is a population of T cells comprising CD27+ cells expressing a recombinant T cell receptor, wherein least 30%, 40%, 60%, 70%, 80%, 85%, 90%, 95% or more of the cells are CD27+ cells. In various embodiments: the population comprises no more than 25%, 20%, 15%, 10%, or 5% CD14+ T cells; and the population receptor comprises no more than 25%, 20%, 15%, 10%, or 5% CD25+ T cells.

In some cases, the CAR T cells are expanded in the presence of exogenously added IL-15 (e.g., at least 10 ng/ml) and minimal or no exogenously added IL-2 (e.g., less than 50 U/ml, less than 40 U/ml, less than 30 U/ml, less than 20 U/ml, less than 10 U/ml, less than 5 U/ml or even less than 1 U/ml) and minimal or no exogenously added IL-7 (e.g., less than 10 ng/ml, less than 8 ng/ml, less than 6 ng/ml, less than 5 ng/ml, less than 3 ng/ml or even less than 1 ng/ml). In some cases, the cells are expanded in the presence of exogenously added IL-15 (e.g., at least 10 ng/ml) and exogenously added IL-2 (e.g., 50 U/ml, 40 U/ml, 30 U/ml, 20 U/ml, 10 U/ml, 5 U/ml IL-2), minimal or no exogenously added IL-7 (e.g., less than 10 ng/ml, less than 8 ng/ml, less than 6 ng/ml, less than 5 ng/ml, less than 3 ng/ml or even less than 1 ng/ml) and minimal or no exogenously added IL-21 (e.g., less than 10 ng/ml, less than 8 ng/ml, less than 6 ng/ml, less than 5 ng/ml, less than 3 ng/ml or even less than 1 ng/ml). In some cases, the only exogenously added interleukin is IL-15 (preferably human IL-15). In some cases, the only exogenously added interleukins are IL-15 (preferably human IL-15) and IL-2. In some cases, all exogenously added interleukins other than IL-15 (e.g., IL-7, IL-21, IL-4 and IL-9) are present at less than 10 ng/ml (less than 8 ng/ml, less than 6 ng/ml, less than 5 ng/ml, 3 ng/ml or even less than 1 ng/ml) and exogenously added IL-2 is present at less than 50 U/ml (less than 40 U/ml, less than 30 U/ml, less than 20 U/ml, less than 10 U/ml, less than 5 U/ml or even less than 1 U/ml). Exogenously added interleukins are those that are added to the culture media as opposed to being generated by the cells themselves.

The T cell populations that can be expanded using the manufacturing methods described herein can include: naïve T cells (T_{N}), memory stem cells (T_{SCM}), central memory T cells (T_{CM}) and combinations thereof in addition to other cells such as effector T cells (T_{E}) or effector memory T cells (T_{EM}). FIG. 1 schematically depicts these cells type and certain of the cell surface markers expressed by each. T cell populations that are primarily naïve T cells (T_{N}), memory stem cells (T_{SCM}), and central memory T cells (T_{CM}) with few T_{E} and T_{EM} cells can be described as T_{CM/SCM/N} cells or T_{MEM/N} cell populations. These cell populations can be derived from peripheral blood mononuclear cells (PBMC) by: 1) depleting unwanted cell populations such as CD14 expressing myeloid cells and CD25 expressing cells; and (optionally) 2) enriching for CD62L expressing memory and naïve T cells. Thus, the resulting population of cells includes T naïve (T_{N}) and stem memory cells (T_{SCM}) expressing CD45RA and CD62L. It also includes the population of central memory T cells (T_{CM}) that express CD45RO and CD62L. T_{MEM/N} cell populations differ from previously described T_{CM} cell populations in that their preparation does not entail depletion of CD45RA+ T cells. T_{MEM/N} cell populations, upon preparation, have only a low level of or are relatively free of effector memory cells (T_{EM}) and effector cells (T_{E}). In addition, such T cell populations have a relatively high proportion or CD45RA+CD45RO− T cells.

In certain embodiments of the manufacturing method described herein, a population of T cells, e.g., a T_{MEM/N} cell population, a T_{CM} cell population, a T_{N} cell population or unselected PBMC, is stimulated and transduced with a vector expressing a desired recombinant T cell receptor, e.g., a CAR. After transduction, the cells are expanded by culturing in a medium comprising exogenously added IL-15 at greater than or equal to: 1 ng/ml, 2 ng/ml, 3 ng/ml, 5 ng/ml or 10 ng/ml

The manufacturing methods described herein can be used to expand T cell populations for a variety of therapeutic purposes. For example, the methods can be used to expand tumor infiltrating lymphocytes (TIL) isolated from a patient.

The manufacturing methods described herein can be used to expand a T cell population that is subsequently transfected with an RNA (e.g., an mRNA) encoding a T cell receptor (Krug et al. 2014 Cancer Immunology and Immunotherapy 63:999)

Patient-specific, autologous and allogeneic T cells (e.g., autologous or allogenic T_{MEM/N} cells) can be engineered to express a chimeric antigen receptor (CAR) or T cell receptor (TCR) and the engineered cells can be enriched for cells expressing CD27.

Described herein is a method for preparing a population of human cells comprising T cells (i.e., CD3+ cells) optionally harboring a recombinant nucleic acid molecule encoding a T cell receptor, comprising: (a) providing a sample of human cells comprising T cells, wherein the T cells comprise: central memory T cells; memory stem T cells, and naïve T cells, wherein greater than 40% (greater than 45%, 50%, 55%, 60%, 65% or 70%) of the T cells are CD45RA+ and greater than 70% (greater than 75%, 80%, 85% or 90%) of the T cells are CD62L+; (b) activating the population of human cells comprising T cells; and (c) transducing or transfecting cells in the population of human cells comprising T cells with a recombinant nucleic acid molecule to provide a population of human cells comprising T cells harboring a recombinant nucleic acid molecule and then treating the transfected cells to enrich for cells expressing CD27.

In various embodiments: the recombinant nucleic acid molecule is a viral vector (e.g., a lentiviral vector or a retroviral vector encoding a T cell receptor such as a CAR); the method further comprises culturing the population of human cells comprising T cells harboring a recombinant nucleic acid molecule; the culturing step comprises the addition of exogenous IL-2 and exogenous IL-15 (and, optionally, little or no exogenously added IL-7 or IL-21); and the activating step comprises exposing the cells to an anti-CD3 antibody and an anti-CD28 antibody; and at least 80% (greater than 85%, 90%, 95%, or 98%) of the cells in the isolated population of cells comprising T cells are T cells. Described herein is method for preparing a population of human cells comprising T cells (i.e., cells that express CD3 or CD3+ cells), wherein the T cells comprise central memory T cells; memory stem T cells, and naïve T cells, wherein greater than 40% (greater than 45%, 50%, 55%, 60%, 65% or 70%) of the cells are CD45RA+ and greater than 70% (greater than 75%, 80%, 85% or 90%) are CD62L+, comprising: (a) providing an isolated population of human cells comprising T cells; (b) treating the isolated population of human cells comprising T cells to deplete cells expressing CD25 and cells expressing CD14 to prepare a depleted cell population; and (c) treating the depleted cell population to enrich for cells expressing CD27 thereby preparing a population of human cells comprising T cells, wherein the T cells comprise central memory T cells; memory stem T cells, and naïve T cells, wherein greater than 40% (greater than 30%, 35%, 40%, 45%, 50%, 55%, 60%, 65%, 70%, 80%, 85%, 90% or 95%) of the cells are CD27+. In some cases, the method does not comprise a step of depleting cells expressing CD45RA.

In some cases, the population of T cells expanded in culture media that includes exogenously added IL-15 and added IL-2 (and, optionally, little or no exogenously added IL-7 or IL-21) can be a population of human cells comprising T cells (i.e., cells that express CD3 or CD3+ cells), wherein the T cells comprise central memory T cells; memory stem T cells, and naïve T cells, wherein greater than 40% (greater than 45%, 50%, 55%, 60%, 65% or 70%) of the cells are CD45RA+ and greater than 70% (greater than 75%, 80%, 85% or 90%) are CD62L+, wherein the population is prepared by a method comprising: providing an isolated population of human cells comprising T cells (e.g. PBMC from a donor); treating the isolated population of human cells comprising T cells to deplete cells expressing CD25 and deplete cells expressing CD14 to prepare a depleted cell population; and treating the depleted cell population to enrich for cells expressing CD62L, thereby preparing a population of human cells comprising T cells, wherein the T cells comprise central memory T cells; memory stem T cells, and naïve T cells, wherein greater than 40% (greater than 45%, 50%, 55%, 60%, 65% or 70%) of the cells are CD45RA+ and greater than 70% (greater than 75%, 80%, 85% or 90%) are CD62L+, wherein the method does not comprise a step of depleting cells expressing CD45RA. In various embodiments: less than 15% (less than 12%, 10%, 8%, 6%) of the T cells in the population of human cells are CD14+ and less than 5% (less than 4%, 3% or 2%) of the T cells are CD25+; at least 40% (greater than 45%, 50%, 55%, 60%, 65% or 70%) of the T cells are CD4+ and CD62L+ or CD8+ and CD62L+; at least 10% (greater than 15%, 20%, 25%, 30%, 35%, or 40%) of the T cells are CD8+ and CD62L+; less than 60% (less than 55%, 50%, 45%, 40%, 35%, 30%, 24%, 20% or 15%) of the T cells are CD45RO+. The population of T cells can be primarily CD4+ cells (greater than 60, 70, 80 or 90% CD4+ cells) or primarily CD8+ cells (greater than 60, 70, 80 or 90% CD8+ cells).

Also described herein is a method of treating cancer, autoimmunity or infection comprising administering to a patient in need thereof a pharmaceutical composition comprising a human T cell population manufactured as described herein. Also described herein is a method of treating cancer, autoimmunity or infection comprising administering to a patient in need thereof a pharmaceutical composition comprising a human T cell population comprising a recombinant T cell receptor wherein at least 15%, 20%, 25%, 30%, 35%, 40%, 50%, 60%, 70%, 80%, 90%) of the T cells are CD27+.

It should be understood that when a cell population is depleted for a particular type of cell, for example, CD14+ and/or CD25+ cells, it does not mean that all such cells are removed from the cell population. In many cases some percentage of such cells remain in the depleted cell population for example less than 15% (12%, 10% 5%, 4%, 3%, 2% or 1%, 5%-1%, 3%-1%) of the cells in the depleted cell population bear the cell marker, e.g., CD14 or CD25. It should also be understood that the percentage of cells bearing a cell marker, e.g., CD14 or CD25, can increase as the cell population is expanded (cultured). Thus, overtime, the percentage of cells bearing the marker can increase to, e.g., 5% or less, 10% or less, or even 15% or less.

In some cases, the cells are autologous to the patient being treated and in some cases they are allogenic to the patient being treated.

### BRIEF DESCRIPTION OF THE DRAWINGS

FIG. 1 depicts certain marker expression data for various T cell subsets.

FIG. 2 depicts the result of a series of studies showing that CD27 mark cells with potent tumor-targeting activity. A) Schema of CAR T cell production process. (B) Isolation and CAR expression on CD27+ and CD27− CAR T cells. (C) Cytotoxicity and expansion capacity against GBM cell rechallenge in vitro. (D) Antitumor efficacy against orthotopic GBM xenografts. (E-F) Activation potential against low antigen expressing GBM cells. (G) Immuno-stimulatory cytokine secretion against in vitro GBM cell stimulation. (H) Engineering T cells to co-express CAR and GFP. T cells were sorted for CAR+, and then GFP/CD27 expression. GFP will be used to distinguish the two subsets post tumor stimulation (I) 1:1 mixed CD27+GFP+/CD27−GFP− CAR T cells were stimulated with tumor cells and evaluated for CAR expansion.

FIG. 3 depicts the results of a series of studies that demonstrate that CD27+ CAR T cells maintain effector function after tumor stimulation. (A) Schema of in vivo CAR T cell stimulation. (B) Recovery of CAR T cells from GBM xenografts. (C) Expression of T cell exhaustion markers in stimulated CAR T cells. (D) Activation potential of in vivo stimulated CAR T cells against re-stimulation. (E) Top upstream regulators comparing CD27+ and CD27− CAR T cells (F) IL2- and IL15-responsive transcriptional signatures in stimulated CAR T cells.

FIG. 4 depicts the results of a series of studies showing that CD27+ CAR T cells interact with CD70 to mediate effector potency. (A) Engineering GBM cells to express targeted antigen at different densities, (B) CAR T cell cytotoxicity/expansion against GBM cells with CD70 blocking antibody, (C) Expression of T cell exhaustion markers when stimulated with PBT103b-EF1α, (D) Detection of soluble CD27 in the co-culture media, (E) CD27 mRNA expression on PBT103b-EF1α stimulated CAR T cells, (F) Activation potential against low antigen expressing targets with CD70 blockades.

FIG. 5 Depicts the result of a series of studies showing that CD70-expressing T cells are responsive to IL-2 mediated ex vivo expansion and loss of memory signature. (A) CD27, CD70 and CD25 (IL2RA) expression on CAR T cells expanded for 14 days. (B) CD27+ and CD27− CAR T cells were isolated at D14 after CAR transduction, and evaluated for expansion in sorted or mixed cultures. (C) Ki67 expression on CD27+ and CD27− CAR T cells. (D-E) Surface marker expression of memory and exhaustion markers on pre-gated CD25+ and CD25− CAR T cells. (F) Expansion of 1:1 mixed CD27+/CD27− CAR T cells cultured with CD70 blockade. (G) Sorted D14 CD27+ CAR T cells were plated for extended culture, and evaluated for CD27/CD70 expression.

FIG. 6 Depicts the results of a series of studies showing that constitutive CD27 signaling inhibits CAR T cell potency. (A-B) Diagram and detection of CAR with constitutively expressed CD27. (C) Cytotoxicity and expansion of CAR T cells with constitutive CD27. (D) Recovery of CAR T cells post in vivo stimulation. (E) Antitumor effect against orthotopic GBM xenografts. (F) Cytotoxic effect against low antigen expressing GBM cells.

FIG. 7 Depicts the results of a series of studies showing that constitutive CD27 induces CAR T cell differentiation and apoptosis. (A) Activation potential of CAR T cells against low antigen expressing GBM cells. (B) Cleaved Caspase-3 staining on CAR T cells stimulated with high antigen expressing GBM cells. (C-D) Expression of memory and exhaustion associated markers in CAR T cells. (E) mRNA expression of memory and exhaustion associated factors in CAR T cells.

### DETAILED DESCRIPTION

The T cell compartment includes T cell subsets that are at different stages of differentiation. These subsets arise from differentiation of Naïve T cells (T_{N}), which are CD45RA+, CD62L+, CD28+, and CD95−. Among the stem cell-like subsets are Memory Stem Cells (T_{SCM}), which are CD45RA+, CD62L+, CD28+, and CD95+. These cells differentiate into Central Memory Cells (T_{CM}), which are CD45RO+, CD62L+, CD28+, and CD95+. T_{CM} differentiate in Effector Memory Cells (T_{EM}), which are CD45RO+, CD62L−, CD28+/−, and CD95+. The T_{EM} differentiate to Effector T cells (T_{E}) which are CD45RO+, CD62L+, CD28+, and CD95+. Beyond these T cell subsets that are different stages of differentiation, there are subsets that either express or do not express various cell surface receptors, e.g., CD14, CD25, CD27 and others. The effectiveness of recombinant T cell receptor based therapy can depend on the nature of the T cells expressing the recombinant T cell receptor, with certain T cell subset being more effective than others.

Human CD27 (GenBank NP_001233; NCBI Gene ID: 939) is a member of the TNF-receptor superfamily and is required for generation and long-term maintenance of T cell immunity. Its ligand is CD70. CD27 plays a key role in regulating B-cell activation and immunoglobulin synthesis. This receptor transduces signals that lead to the activation of NF-kappaB and MAPK8/JNK. Adaptor proteins TRAF2 and TRAF5 have been shown to mediate the signaling process of this receptor. CD27-binding protein (SIVA), a proapoptotic protein, can bind to this receptor and is thought to play an important role in the apoptosis induced by this receptor.

Described herein are studies showing that recombinant T cells that are CD27+ have desirable properties for use in recombinant T cell therapy. Thus, it is desirable to prepare T cell populations that are enriched for CD27+ T cells. The enrichment can be performed before or after introduction into the T cells of a recombinant nucleic acid molecule expressing a recombinant T cell receptor (e.g., a CAR).

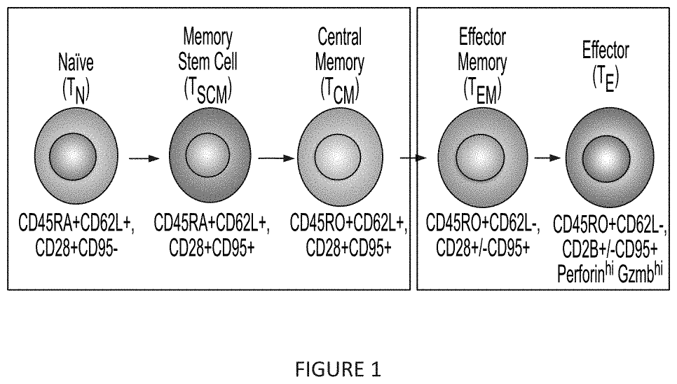

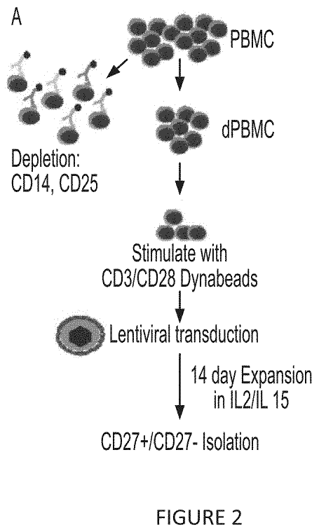

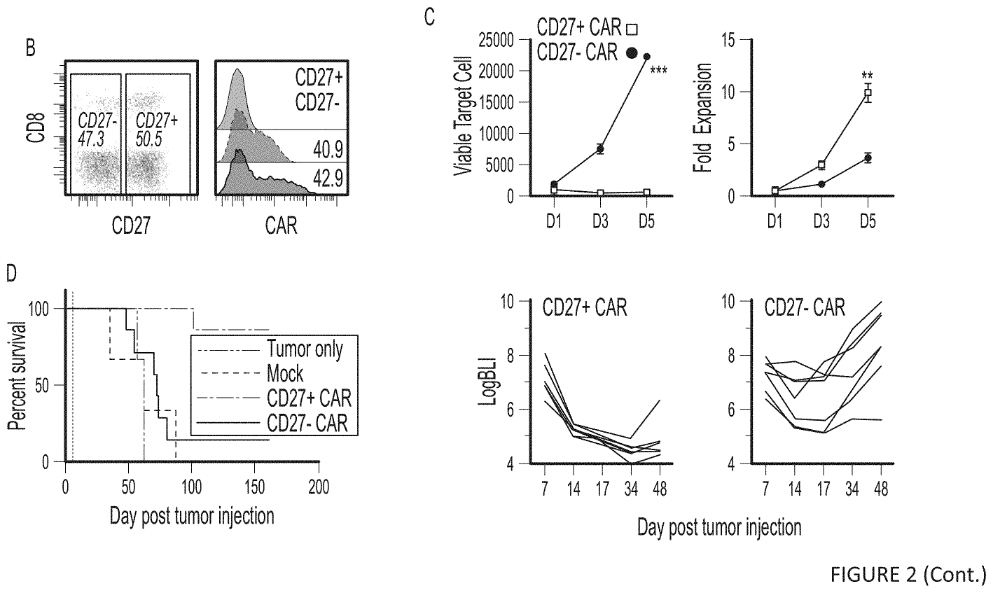

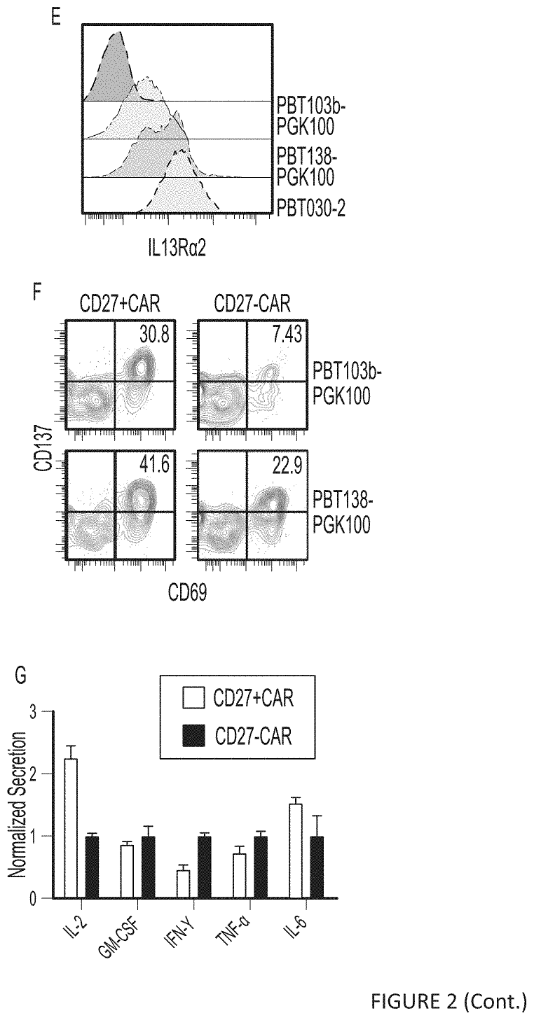

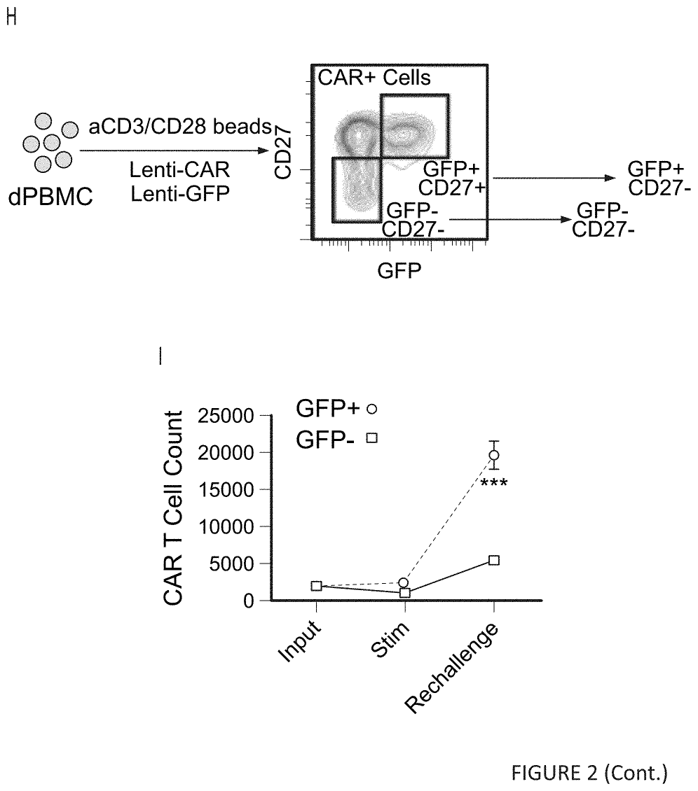

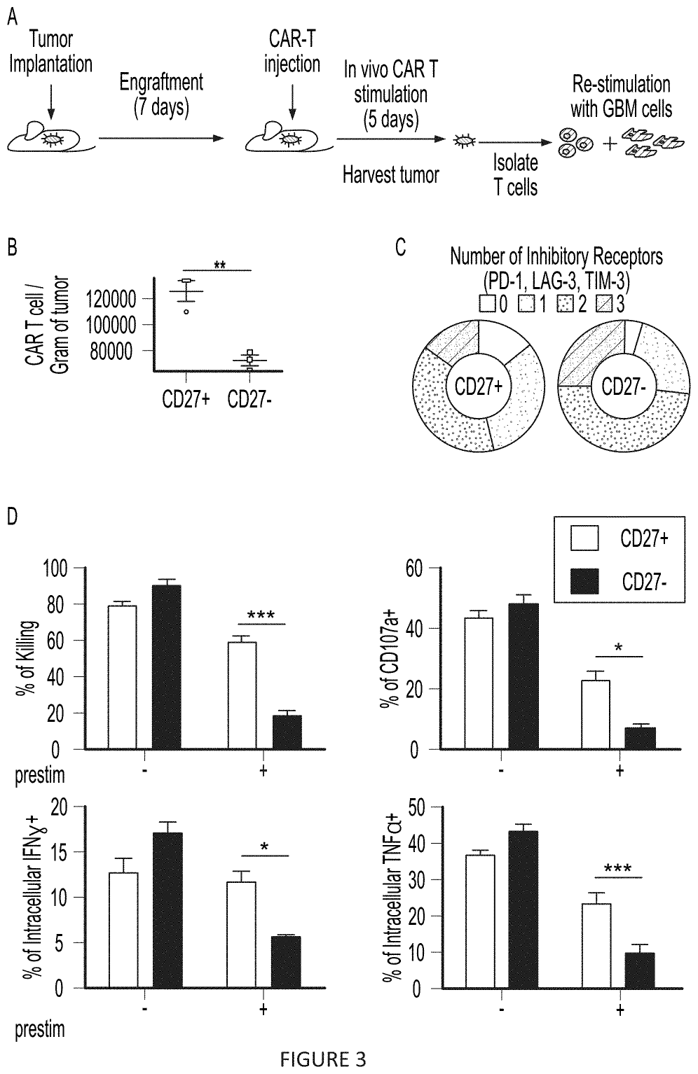

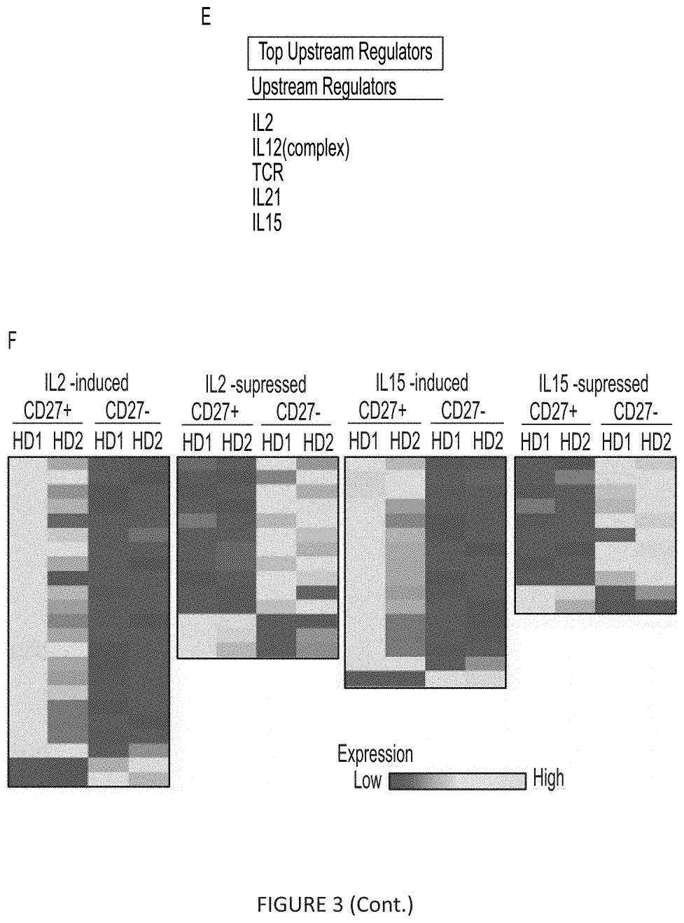

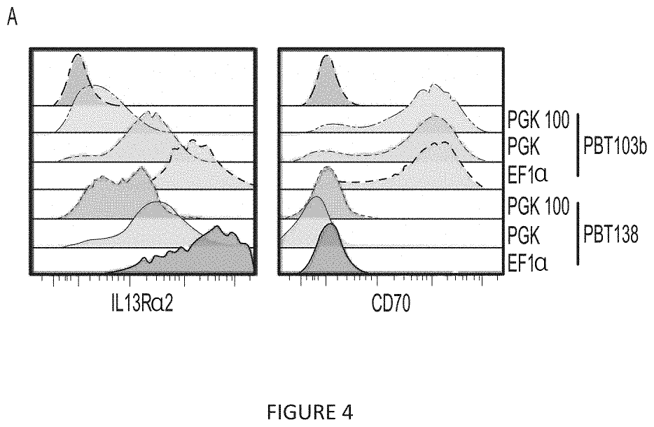

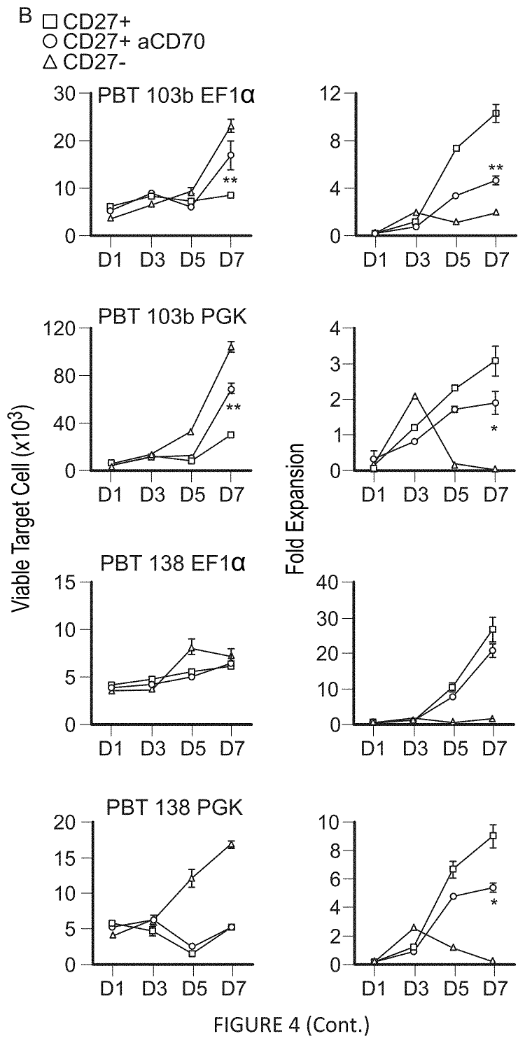

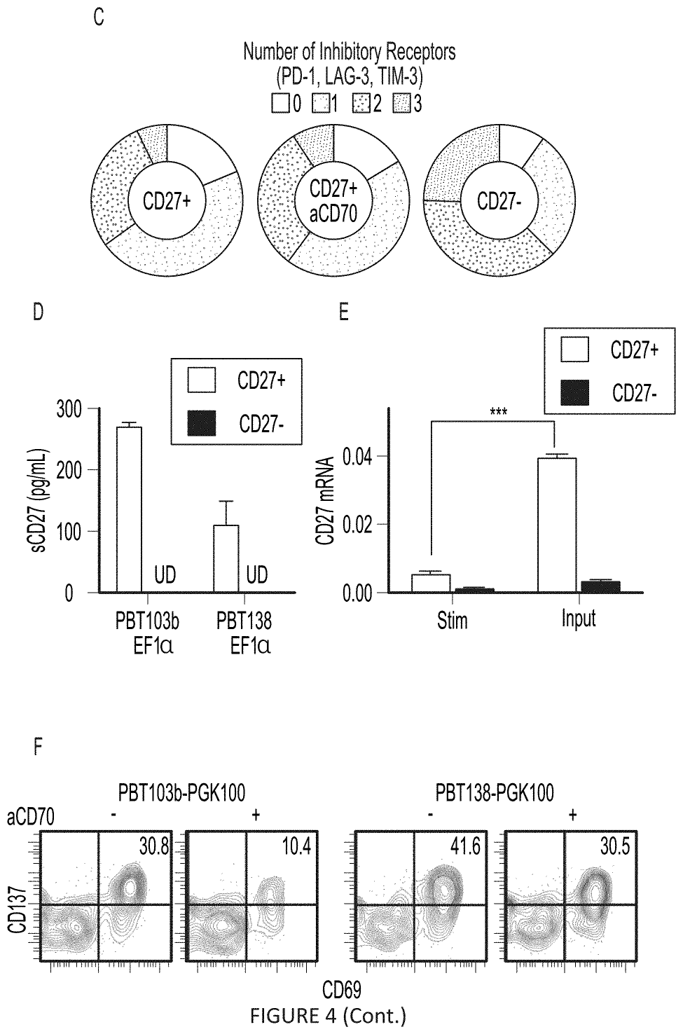

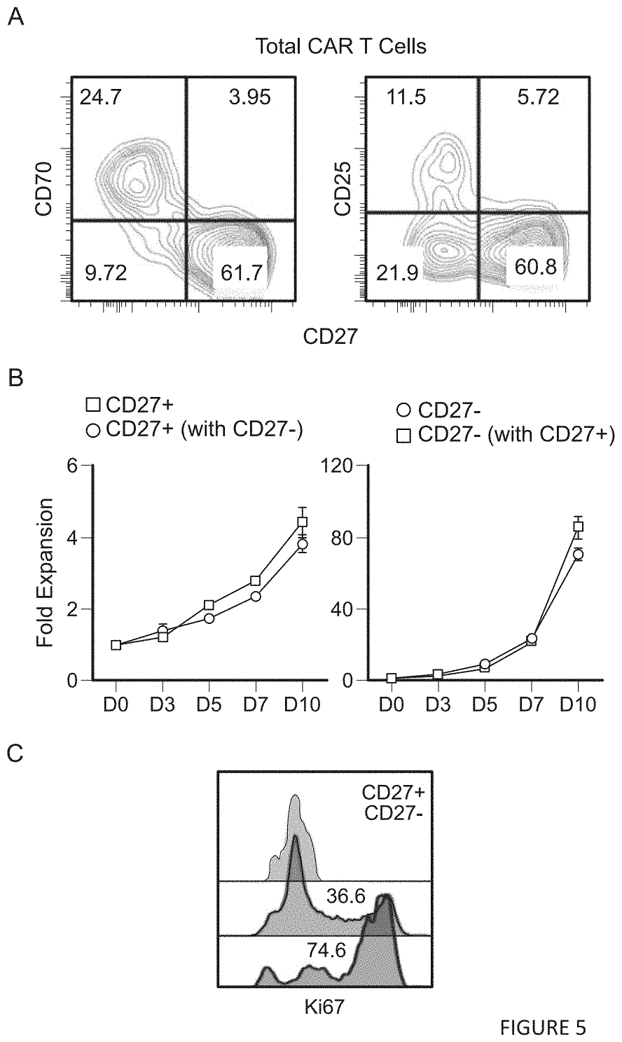

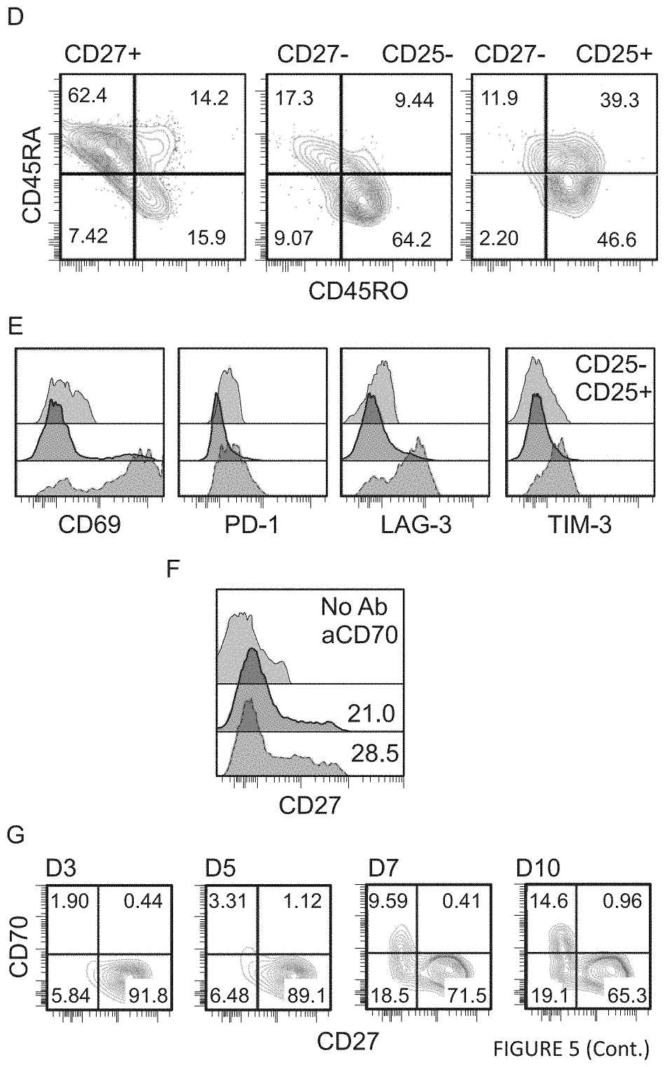

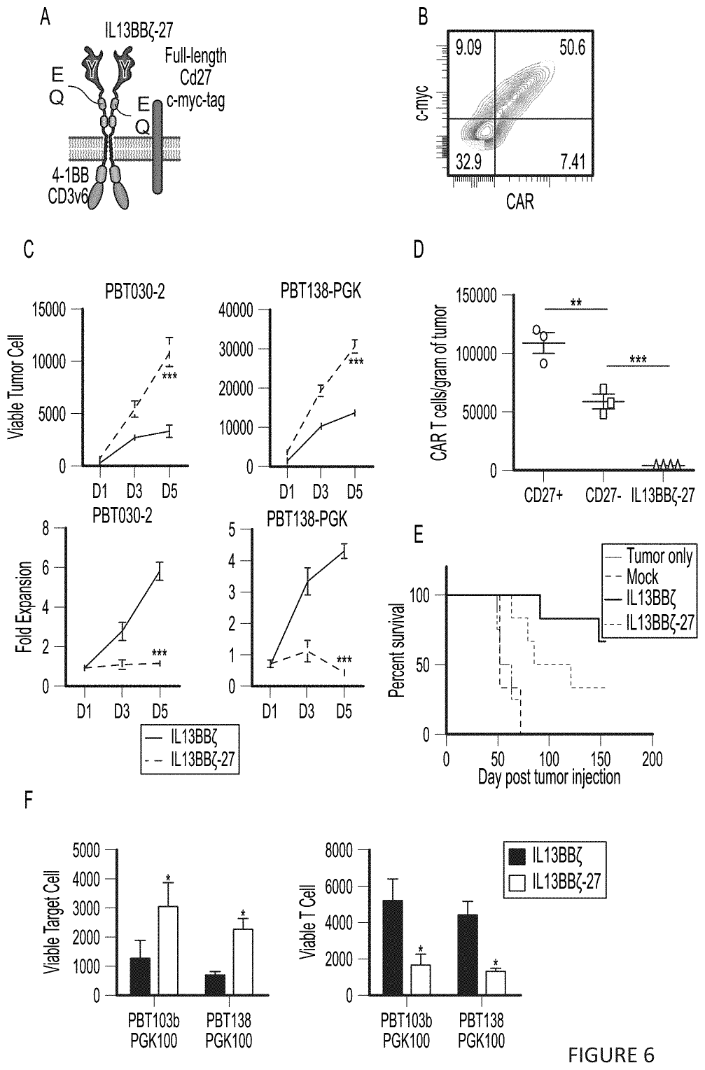

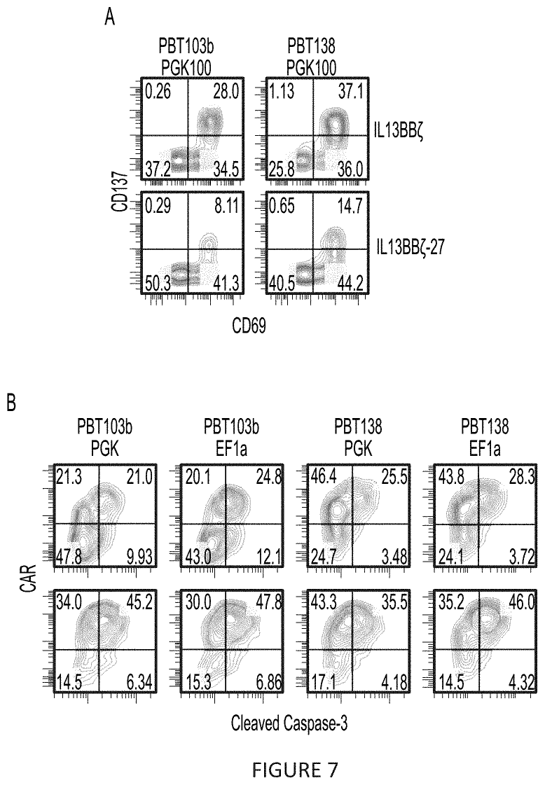

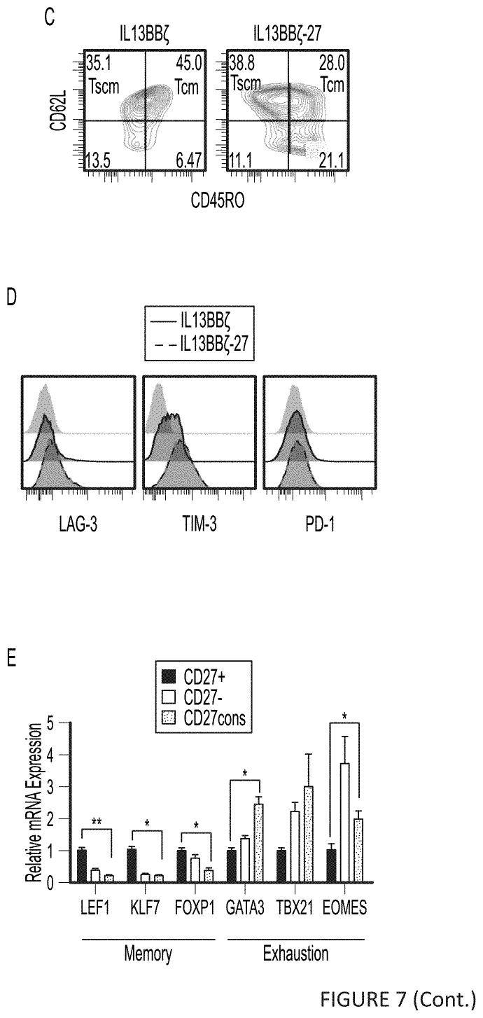

Memory Stem T Cells (T_{SCM}) are present at a low level in the T cell compartment, but appear to have significant self-renewal and proliferative potential. While they resemble naïve T cells (T_{N}) in that they express CD45RA+ and CD62L+, they can be distinguished from T_{N} by their expression of CD95 (FIG. 1). T_{SCM} can be generated from T_{N} by stimulation with CD3/CD28 beads in the presence of IL-7 and IL-15. They also can be expanded in the presence of Wnt/β-catenin pathway activation (Cieri et al. 2013 Blood 121:573; Gattinoni et al. 2009 Nature Medicine 15:808).

Central Memory T Cells (T_{CM}), which are more abundant in PBMC than are T_{SCM}, are a well-defined memory T cell subset with high self-renewal and proliferative potential. There is evidence that T_{CM} persist following adoptive transfer better than Effector T cells (T_{E}) (Berger et al. 2008 Journal of Cellular Immunology 118:4817; Wang et al 2011 Blood 117:1888). T_{CM} can be enriched from PBMC for T cell therapy manufacturing based on their CD45RA− CD45RO+CD62L+ phenotype (Wang et al. 2012 J Immunotherapy 5:689). There is some evidence that T_{CM} behave as adult stem cells. Studies in mice demonstrated that: single cell transfer of T_{CM} over three generations demonstrated that T_{CM} can provide full immune reconstitution; that T_{CM} expand to produce more T_{CM}; and that T_{CM} differentiate to T_{EM}/T_{E} (Graef et al. 2014 Immunity 41:116; Gattioni et al. 2014 Immunity 41:7).

The various T cell populations described can be genetically engineered to express, for example, a CAR or a T cell receptor. A CAR is a recombinant biomolecule that contains an extracellular recognition domain, a transmembrane region, and one or more intracellular signaling domain. The term “antigen,” therefore, is not limited to molecules that bind antibodies, but to any molecule that can bind specifically to any receptor. “Antigen” thus refers to the recognition domain of the CAR. The extracellular recognition domain (also referred to as the extracellular domain or simply by the recognition element which it contains) comprises a recognition element that specifically binds to a molecule present on the cell surface of a target cell. The transmembrane region anchors the CAR in the membrane. The intracellular signaling domain comprises the signaling domain from the zeta chain of the human CD3 complex and optionally comprises one or more co-stimulatory signaling domains. CARs can both to bind antigen and transduce T cell activation, independent of MHC restriction. Thus, CARs are “universal” immunoreceptors which can treat a population of patients with antigen-positive tumors irrespective of their HLA genotype. Adoptive immunotherapy using T lymphocytes that express a tumor-specific CAR can be a powerful therapeutic strategy for the treatment of cancer.

The CAR can be produced by any means known in the art, though preferably it is produced using recombinant DNA techniques. Nucleic acids encoding the several regions of the chimeric receptor can be prepared and assembled into a complete coding sequence by standard techniques of molecular cloning known in the art (genomic library screening, overlapping PCR, primer-assisted ligation, site-directed mutagenesis, etc.) as is convenient. The resulting coding region can be inserted into an expression vector and used to transform a suitable expression host cell line, preferably a T lymphocyte cell line, and most preferably an autologous T lymphocyte cell line. Alternatively, the coding region can be transiently expressed by an RNA that is introduced into the T cells after expansion using the methods described herein.

The nucleic acid molecule encoding a CAR can encode a CAR that comprises an scFv directed to a target, e.g., CD19, IL13Ra, PSCA, HER2, MCL, BMCA, TAG72, a spacer, a transmembrane domain (e.g., CD4, CD8 or CD28 TM domain), at least one co-stimulatory domain (e.g., CD28, OX040, 4-1BB or ICOS) and a CD3zeta signaling domain.

Various CAR suitable for expression by the cell populations described herein include, for example, those described in: WO 2016/044811; WO 2104/144622; WO 2002/077029; and WO/US2014/0288961.

### Example 1: Preparation of T_{CM/SCM/N} Cells

A variety of methods can be used to produce a population of human T_{CM/SCM/N} cells. For example, a population of T_{CM/SCM/N} cells can be prepared from a mixed population T lymphocytes. The population of T lymphocytes can be allogenic to or autologous to the subject ultimately treated using the cells and can be obtained from a subject by leukopheresis or blood draw.

The following method is an example of one that can be used to obtain a population of T_{CM/SCM/N} cells from T lymphocytes obtained by leukapheresis or other means. Peripheral blood is collected by leukapheresis or peripheral blood draw. Day 1 of a typical manufacturing cycle is the day the ficoll procedure takes place. The subject's leukapheresis product is diluted with EDTA/PBS and the product is centrifuged at 1200 RPM for 10 minutes at room temperature with maximum brake. After centrifugation, the platelet-rich supernatant is removed and the cell pellet is gently vortexed. EDTA/PBS is used to re-suspend the vortexed cell pellets in each conical tube. Each tube is then underlayed with ficoll and centrifuged at 2000 RPM for 20 minutes with no brake at room temperature. Following centrifugation, the PBMC layer from each tube is transferred into another conical tube. The cells are centrifuged at 1800 RPM for 15 minutes with maximum brake at 4° C.

After centrifugation, the cell-free supernatant is discarded and the cell pellet is gently vortexed. The cells are washed twice using EDTA/PBS each time, and a third time using PBS. Cells are centrifuged each time at 1200 RPM for 10 minutes with maximum brake at 4° C. After the final PBS wash, the vortexed cell pellet is resuspended in complete X-VIVO 15 media (X-VIVO™ media with 10% FBS) and transferred to a transfer bag. The bag with washed PBMC is kept overnight on a rotator at room temperature on the bench top for immunomagnetic selection the next day.

Next, selection procedures are used to both to deplete the cell population of cells expressing certain markers and to enrich the cell population for cells expressing certain other markers. These selection steps preferably occur on day two of the manufacturing cycle. The cell population is substantially depleted for cells expressing CD25 and CD14. Preferably, the cell population is not substantially depleted for cells expressing CD45RA. Briefly, cells resuspended in labeling buffer (LB; EDTA/PBS with 0.5% HSA), and incubated with anti-CD14 and anti-CD25 i antibodies or antibody coated beads for CliniMACS® depletion (Miltenyi Biotec), and the composition is gently mixed and then incubated for 30 minutes on a rotator at room temperature on the bench top.

The depletion step is performed on a CliniMACS® device using a depletion tubing set. The recovered cells following the depletion step are transferred into tubes and centrifuged at 1400 RPM for 15 minutes with maximum brake at 4° C.

The cell-free supernatant is removed and the cell pellet is gently vortexed and resuspended. To enrich for cells expressing CD27, the cell suspension is can be treated with anti-CD27-biotin or sorted using beads coated with anti-CD27 antibodies (Miltenyi Biotec).

Following the incubation period, LB is added to the tube and cells are centrifuged at 1400 RPM for 15 minutes at maximum brake at 4° C. The cell-free supernatant is removed and the cell pellet is gently vortexed. LB is added to resuspend the cell pellet in the tube and the resuspended cells are transferred to a new transfer bag. Anti-biotin (Miltenyi Biotec) reagent is added and the mixture is gently The CD27 enrichment step can be performed on a CliniMACS® device using a tubing set. The product of this enrichment can be frozen for storage and later thawed and activated

To provide an intermediate holding step in the manufacturing, the option exists to freeze cells following the selection process. The cells are pelleted by centrifugation at 1400 RPM for 15 minutes with max break at 4° C. The cells are resuspended in Cryostor® and aliquoted into cryovials. The vials are transferred to a controlled cooling device that can cool at about 1° C./minute (e.g., a Nalgene® Mr. Frosty; Sigma-Aldrich) the cooling device is immediately transferred to a −80° C. freezer. After three days in the −80° C. freezer, the cells are transferred into a GMP LN2 freezer for storage.

Cryopreserved cells can exhibit good recovery and viability, maintain the appropriate cell surface phenotype when thawed up to 8.5 months after cryopreservation, and can be successfully transduced and expanded in vitro upon thawing.

Alternatively, freshly enriched T_{CM/SCM/N} cells can be activated, transduced and expanded as described below.

### Example 2: Activation, Lentiviral Transfection and Culturing in the Presence of Certain Cytokines

Human T cells, either bulk PBMC or enriched T cell subsets, are stimulated as for example with GMP Dynabeads® Human T expander CD3/CD28 (Invitrogen) at a 1:3 ratio (T cell:bead). On day 0 to 3 of cell stimulation, T cells are transduced, for example with a CAR-expressing lentivirus, in X Vivo 15 containing 10% fetal calf serum (FCS) with 5 g/mL protamine sulfate (APP Pharmaceutical), and with exogenously added cytokines (i.e., final concentration 10 ng/mL rhIL-15 and IL-2). Either before or after transfection the cell population is treated to enrich for cells that express CD27. Following lentivirus transduction, media is exchanged or cultures diluted 1:2 to in X Vivo 15 containing 10% FCS and cytokines. Cultures are then maintained at 37° C., 5% CO₂ with addition of X-Vivo15 10% FCS as required to keep cell density between 3×10⁵ and 2×10⁶ viable cells/mL, with cytokine supplementation (i.e., final concentration of 10 ng/mL rhIL-15) every Monday, Wednesday and Friday of culture. On day 7 to 10 following T cell stimulation, the CD3/CD28 Dynabeads are removed from cultures using the DynaMag-50 magnet (Invitrogen). Cultures are propagated until day 8 to 32 days and then cryopreserved. Over the duration of the culture, cells are supplemented with a combination of cytokines (for example, IL2 (50 U/mL)+IL15 (0.5 ng/mL), IL7 (10 ng/mL)+IL15 (10 ng/mL) or IL7 (10 ng/mL)+IL15 (10 ng/mL)+IL21 (10 ng/mL), or IL-15 only (10 ng/mL)). Two thirds of the culture media is removed and fresh media consisting of above cytokine combination is added at a 0.6×10⁶ cells/mL concentration. Exogenous cytokine addition is optional during the CD3/CD28 bead stimulation phase, however, it is essential during the expansion phase following removal of the beads. The amount of cytokine added to reach a desired level of exogenously added cytokine is based in the assumption that any media not replaced when fresh media is added is essentially free of any previously exogenously added cytokine.

1. A method for preparing a population T cells comprising CD27+ cells expressing a recombinant T cell receptor, comprising:
(a) providing a population of human T cells;
(b) treating the human T cell population to enrich for CD27+ cells thereby providing a population of T cells that is enriched for CD27+ cells relative to the provided human T cell population; and
(c) introducing a nucleic acid molecule expressing a recombinant T cell receptor into the population of cells that are enriched for CD27+ cells relative to the provided T cell population thereby providing population of T cells comprising CD27+ cells expressing a recombinant T cell receptor.

2. A method for preparing a population T cells expressing a recombinant T cell receptor, comprising:
(a) providing a population of human T cells;
(b) introducing a nucleic acid molecule expressing a recombinant T cell receptor into the provided population of human T cells thereby providing a population of T cells expressing a recombinant T cell receptor; and
(c) treating the population of T cells expressing a recombinant T cell receptor to enrich the population for CD27+ cells thereby providing a population of T cells comprising CD27+ cells expressing a recombinant T cell receptor.

3. The method of claim 1, wherein the step of providing a population of human T cells comprises:
(a) providing a population of human T cells;
(b) treating the provided population of human T cells to deplete CD14+ cells and CD25+ cells and thereby providing a population of human T cells; and
(c) optionally, treating the population of human T cells to enrich for CD62L+ cells.

4. The method of claim 1, wherein the step of providing a population of human T cells does not comprise treating the population of human T cells to deplete CD45RA+ cells.

5. The method of claim 1, wherein the step of introducing a nucleic acid molecule comprises prior activation of the cells by exposure to an anti-CD3 antibody and an anti-CD28 antibody.

6. The method of claim 1, wherein the recombinant T cell receptor is a chimeric antigen receptor.

7. The method of claim 1, wherein the population of T cells comprising CD27+ cells expressing a recombinant T cell receptor comprises at least 30%, 40%, 60%, 70% or 80% CD27+ T cells.

8. The method of claim 1, wherein the provided population of human T cells comprises no more than 25%, 20%, 15%, 10%, or 5% CD25+ T cells.

9. The method of claim 1, wherein the provided population of T cells comprises no more than 25%, 20%, 15%, 10%, or 5% CD14+ T cells.

10. The method of claim 1, wherein the provided population of T cells comprises at least 30%, 40%, 50%, 60%, 70% or 80% CD62L+ cells.

11. The method of claim 1, wherein the population of T cells comprising CD27+ cells expressing a recombinant T cell receptor comprises no more than 25%, 20%, 15%, 10%, or 5% CD25+ T cells.

12. The method claim 1, wherein the population of T cells comprising CD27+ cells expressing a recombinant T cell receptor comprises no more than 25%, 20%, 15%, 10%, or 5% CD14+ T cells.

13. A population of T cells prepared by the method of claim 1.

14. The population of T cells of claim 13, wherein at least 30%, 40%, 60%, 70% or 80% of the cells are CD27+ cells.

15. The population of T cells of claim 13, wherein less than 5% of the cells are CD25+ and/or less than 5% of the cells are CD14+.

16. A population of T cells comprising CD27+ cells expressing a recombinant T cell receptor, wherein least 30%, 40%, 60%, 70% or 80% of the cells are CD27+ cells.

17. The population of T cells of claim 16, wherein the population comprises no more than 25%, 20%, 15%, 10%, or 5% CD14+ T cells.

18. The population of T cells of claim 16, wherein the population comprises no more than 25%, 20%, 15%, 10%, or 5% CD25+ T cells.

19. The method of claim 1, wherein the recombinant T cell receptor is a chimeric antigen receptor.

20. The population of T cells of claim 13, wherein the recombinant T cell receptor is a chimeric antigen receptor.

21. A method of treating a patient, the method comprising administering a composition comprising a population of T cells comprising CD27+ cells expressing a recombinant T cell receptor, wherein at least 30%, 40%, 60%, 70% or 80% CD27+ T cells.

22. A population of human T cells, wherein at least 30%, 40%, 50%, 60%, 70% or 80% of the T cells comprise a nucleic acid molecule encoding a recombinant T cells receptor and least 30%, 40%, 60%, 70% or 80% of the human T cells are CD27+ T cells.

23.-28. (canceled)
